(19) United States
(12) Patent Application Publication
(10) Pub. No.: US 2021/0315727 A1
(43) Pub. Date: Oct. 14, 2021

(54) PHYSIOLOGICAL FIXED DEVICE AND ASSEMBLY OF PHYSIOLOGICAL FIXED DEVICE AND COLLECTING BAG

(71) Applicant: Jason Jiang, TAICHUNG CITY (TW)

(72) Inventor: Jason Jiang, TAICHUNG CITY (TW)

(21) Appl. No.: 17/230,398

(22) Filed: Apr. 14, 2021

(30) Foreign Application Priority Data
Apr. 14, 2020 (TW) 109204317

(51) Int. Cl. A61F 5/44; A61F 5/451

(52) U.S. Cl. CPC A61F 5/4408; A61F 5/451

(57) ABSTRACT

A physiological fixed device includes a main body unit having a main body respectively provided on top and bottom surfaces thereof with an abutted portion for contacting a human body, which has at least one excretion collecting opening for excretion to fall into a collecting part and V-shaped cross sections corresponding to the physiological structure of the buttocks groove for good fitness, and an accommodating portion for accommodating a urine bag. An underclothing unit is connected with the main body unit for being positioned on the human body to locate the main body unit at the crotch, so that it is convenient for wearing and carrying, suitable for men and females with different physiological structures, useful for collecting feces and urine, and high in compatibility, thereby convenient and practical. This invention also provides an assembly of the aforementioned physiological fixed device and a collecting bag.

### BACKGROUND OF THE INVENTION

### 1. Field of the Invention

The present invention relates to a fixed device and more particularly, to a physiological fixed device which is adapted to collect excretion for a human body by a collecting member provided thereon with a urine bag, and capable of replacement and reuse.

### 2. Description of the Related Art

For postoperative patients, the elderly or those who have limited mobility, they firstly face the physiological problem of excretion. Since the patients cannot go to the toilet for excretion, they need leak-proof supplies or collection utensils to collect the excretion and be replaced.

The commonly used excretion collecting manners can be roughly classified into a leak-proof type and a collection type. The leak-proof type refers to that uses appropriate absorbent supplies or absorbent pads to prevent the excretion of the patient from leaking or oozing. The most common ones are paper diapers for patients to wear. The paper diaper is replaced at regular time or when defecation. For the collection type, it uses a urine basin, urine pot, or a bag or protective bag carried with the patient for collecting the excretion into the urine bag or protective bag, and it is replaced at regular time.

In general, the conventional paper diapers or urine cloths of leak-proof type collect the excretion primarily in a way that it is worn directly on the hip in a planar manner for the excretion to be located in the paper diaper without leakage to avoid contaminating the outer pants. However, such manner will bring uncomfortableness because the excretion is directly in contact with the skin, and it is difficult to clean up when a large area of skin is contaminated. If the patient wears it for a long time or does not change it often, it is liable to cause diaper rash.

The conventional urine basin and urine pot of collection type are placed at a fixed location, such as the bedside. When excreting is needed, the urine basin or urine pot is taken onto the bed or hold by a hand to collect the excretion. The urine bags are designed to be carried around, and most of them have a relatively more supportive collecting member in coordination with a collecting protective bag or urine bag. For advanced ones, the collecting member is connected or sewed on the clothing for the convenience of directly wearing it on the body. After direct defecation, the excretion is guided to the urine bag through a guiding member such as a urine guiding pipe or guiding seat, or the excretion is directly defecated in a urine bag or box disposed at the anus. Such collection type can solve the uncomfortableness of using paper diapers or urine cloth and reduce the generation of diaper rash. Besides, in such excretion collecting structure, the collecting member can be reused, and only the urine bag should be replaced, that can greatly reduce the generation of waste.

For the conventional excretion collecting structure of carry-on collection type, Taiwan Patent No. 78212495 disclosed a urine cover device, which includes a rubber ring, several hook and loop straps with an end thereof fixed to the aforementioned rubber ring, a hook and loop belt passing through the aforementioned several hook and loop straps, a pull strip with an end thereof fixed to the aforementioned rubber ring and another end fixed at an appropriate position of the aforementioned hook and loop belt, a urine bag member with an end portion thereof rolled into the aforementioned rubber ring when the main rubber is in a hot melt state and a urine proof layer provided therein and having a bottom end portion with several urine drawing holes, several urine storing bags, and several connectors with the appearance thereof having male threads, an end thereof inserted in the aforementioned urine drawing hole and another end where the aforementioned urine storing bag is sleeved and fixed by a wire.

There is another conventional excretion collecting structure of carry-on collection type. Taiwan Patent No. 104107298 disclosed a urine collector, a urine receiving device and a sleeve for being connected with a urine collecting bag. The sleeve has an inlet, an outlet, and at least one anti-reflux portion annularly protruding inward from the inner wall of the sleeve and formed with a bend toward the outlet. The outlet of the sleeve is adapted to communicate with the urine collecting bag. In this way, the penis of the male genitalia is inserted into the sleeve from the inlet thereof, and the urine is blocked by the anti-reflux portion to avoid flowing back and introduced into the urine collecting bag through the outlet.

There is still another conventional excretion collecting structure of carry-on collection type. Taiwan Patent No. 104110871 disclosed a feces collecting device, which includes a wear member including a carrier adapted for a patient to wear and having an opening located correspondingly to the anus of the patient when the patient wears the carrier, an anus plug including an elastic ring and detachably combined with the wear member to locate the elastic ring in the opening, and a collecting bag with an outer peripheral wall thereof combined with the inner annular surface of the elastic ring and an inner peripheral wall provided with glue located close to the bag opening. The bag opening of the collecting bag is folded to cover the outer annular surface of the elastic ring, thereby exposing the inner peripheral wall provided with the glue. For the aforementioned feces collecting device, when the aforementioned patient wears the carrier, the glue of the inner peripheral wall of the collecting bag is attached to the inner wall of the anus of the patient.

The aforementioned structures are all excretion collecting devices of carry-on type. The first case is a structure with the function of collecting feces and urine, which uses a bowl-shaped collecting member provided with two urine collecting openings in coordination with two urine collecting bags detachably disposed at the urine collecting openings. The urine collecting device of the second case has a sleeve to serve as the collecting member for corresponding to the male genitalia, and a guiding pipe to guide the urine in the sleeve to a urine collecting bag. The third case primarily has a wear member, and an anus plug is disposed on the wear member and located correspondingly to the anus to serve as the collecting member for a collecting bag to be inserted therein. The collecting bag is provided at two predetermined parts of the bag opening thereof with glue for being fixedly attached to the anus plug and the inner wall of the anus of the human body, respectively.

However, although the collecting members of the above-described conventional cases are supportive and thereby prevented from direct contact with the skin and the cleaning inconvenience or diaper rash it will cause, their shapes do not conform to the structure of the buttocks groove of the human body and have poor fitness and quite volume, making the wearer feel a foreign object, liable to cause friction when walking, and walk with strange posture. Besides, for the manner in the above-described third case that the anus plug is fastened to the inner wall of the anus by glue, the long-term action of tearing off and reattaching is liable to damage the skin of the anus, and the glue may be allergic to the skin and difficult to clean. In addition, the above-described conventional structures mostly have the collecting member adapted for the male genitals for collecting the urine primarily, not only incapable of collecting feces at the same time, but also unsuitable for female physiological structure so as to be incompatible. Moreover, the conventional structures fail to provide a space for accommodating the urine bag more supportively and the back body is liable to be accidentally compressed to cause the excretion therein to leak. Therefore, the present invention proposes a solution in response to the aforementioned conventional problems, providing a carry-on fixed device adapted for collecting excretion and convenient for wearing.

### SUMMARY OF THE INVENTION

The present invention discloses a physiological fixed device which includes a main body unit having a main body provided at a predetermined part thereof with V-shaped cross sections, and the main body is respectively provided on top and bottom surfaces thereof with an abutted portion for contacting a human body and an accommodating portion forming a space. The abutted portion is provided with at least one excretion collecting opening with penetrating shape for excretion to fall into a collecting part. An underclothing unit is connected with the main body unit. The underclothing unit can be positioned on the human body and make the main body unit located at the crotch of the human body. By the aforementioned members, the abutted portion with V-shaped cross sections corresponds to the physiological structure of the buttocks groove and effectively fit, thereby having relatively better fitness, and the accommodating portion is formed for accommodating a urine bag or urine bag space. In coordination with the underclothing unit, it is convenient for wearing and carrying, suitable for males and females with different physiological structures, useful for collecting feces and urine at the same time, and high in compatibility, so the entire structure is convenient and practical.

It is a primary objective of the present invention to provide a physiological fixed device which has an abutted portion with V-shaped cross sections to fit the buttocks groove, thereby relatively better in fitness and ergonomic. It is another objective of the present invention to provide a physiological fixed device which has an accommodating portion with V-shaped cross sections for forming a space to have the portion providing a urine bag or accommodating a urine bag therein, thereby having the accommodating function. It is still another objective of the present invention to provide a physiological fixed device which can be combined with an underclothing unit to attain the effect of convenience for wearing and carrying. It is a further objective of the present invention to provide a physiological fixed device which is suitable for males and females with different physiological structures and useful for collecting feces, urine and menstrual blood at the same time, thereby having high compatibility and good practicality.

The present invention further provides an assembly of the aforementioned physiological fixed device and a collecting bag, wherein a collecting bag is disposed at the excretion collecting opening for collecting the excretion therein.

### BRIEF DESCRIPTION OF THE DRAWINGS

FIG. 1 is a perspective view showing the appearance of the main body unit of a preferred embodiment of the present invention.

FIG. 2 is another perspective view showing the appearance of the main body unit of a preferred embodiment of the present invention.

FIG. 3 is a planar view of the main body unit of a preferred embodiment of the present invention.

FIG. 4 is a sectional view of the main body unit of a preferred embodiment of the present invention.

FIG. 5 is a cross-sectional view of the main body unit of a preferred embodiment of the present invention.

FIG. 6 is another cross-sectional view of the main body unit of a preferred embodiment of the present invention.

FIG. 7 is a perspective view showing a use state of a preferred embodiment of the present invention combined with the underclothing unit.

FIG. 8 is another perspective view showing the use state of a preferred embodiment of the present invention combined with the underclothing unit.

FIG. 9 is a schematic view of a preferred embodiment of the present invention assembled with a collecting bag.

FIG. 10 is another schematic view of a preferred embodiment of the present invention assembled with the collecting bag.

FIG. 11 is a schematic view showing a use state of a preferred embodiment of the present invention and the buttocks of the human body.

FIG. 12 is a schematic perspective view showing the appearance of a second embodiment of the present invention.

FIG. 13 is a planar view of the main body unit of the second embodiment of the present invention.

FIG. 14 is another planar view of the main body unit of the second embodiment of the present invention.

FIG. 15 is a perspective view showing the appearance of a third embodiment of the present invention.

FIG. 16 is a schematic view showing a use state of a fourth embodiment of the present invention.

FIG. 17 is a schematic perspective view showing the appearance of the collecting bag of a fifth embodiment of the present invention.

FIG. 18 is an assembled schematic view of the fifth embodiment of the present invention.

FIG. 19 is another assembled schematic view of the fifth embodiment of the present invention.

FIG. 20 is a planar view of the main body unit of a sixth embodiment of the present invention.

FIG. 21 is another planar view of the main body unit of the sixth embodiment of the present invention.

FIG. 22 is a perspective view showing a use state of the sixth embodiment of the present invention combined with the underclothing unit.

FIG. 23 is a cross-sectional view of the main body unit of the sixth embodiment of the present invention.

FIG. 24 is a schematic view showing a use state of the collecting bag of the sixth embodiment of the present invention.

FIG. 25 is a cross-sectional view of a seventh embodiment of the present invention.

FIG. 26 is a schematic view showing a use state of the collecting bag of an eighth embodiment of the present invention.

FIG. 27 is a schematic view showing the installation of the collecting bag of the eighth embodiment of the present invention.

### DETAILED DESCRIPTION OF THE INVENTION

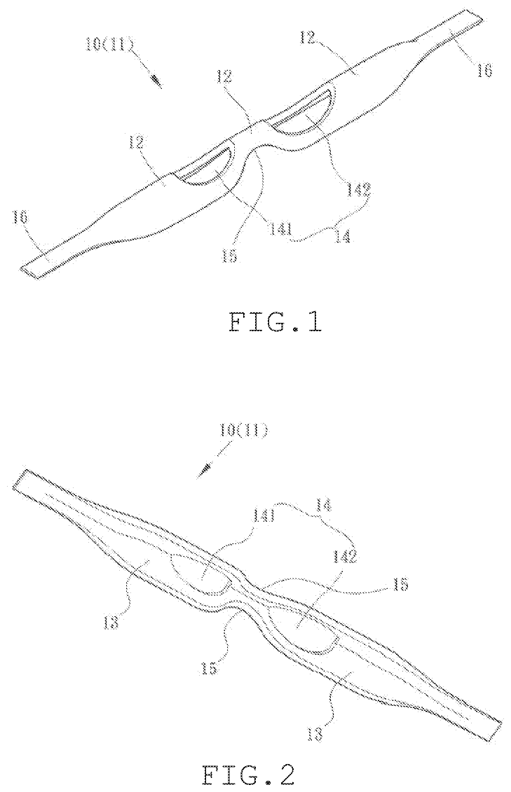

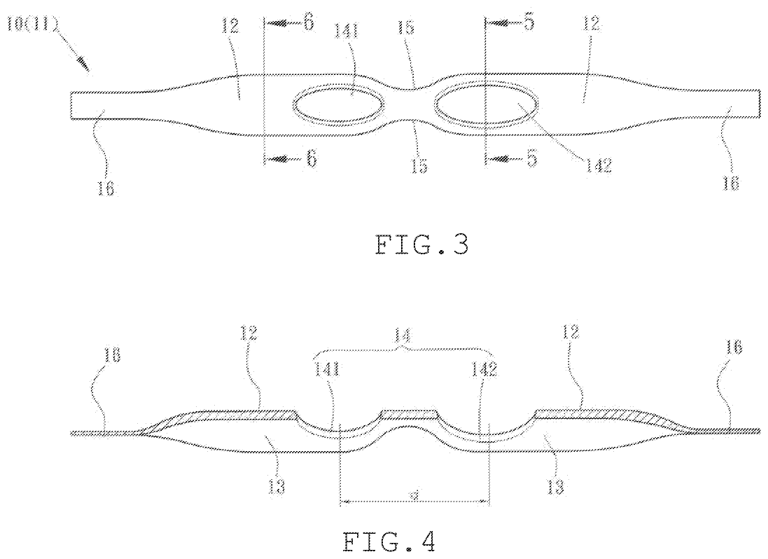

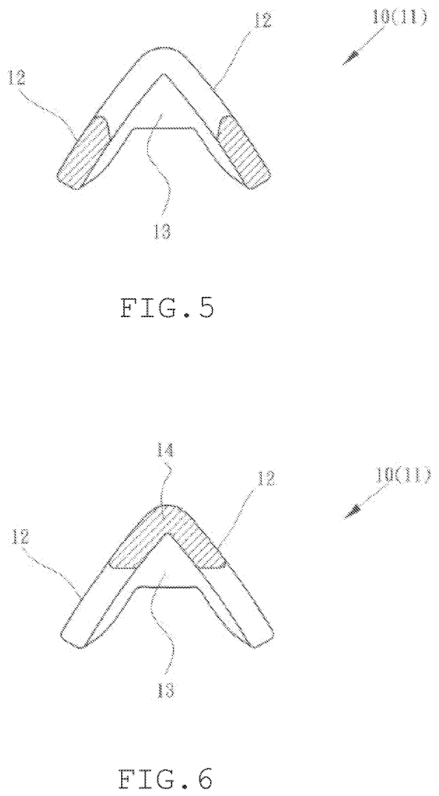

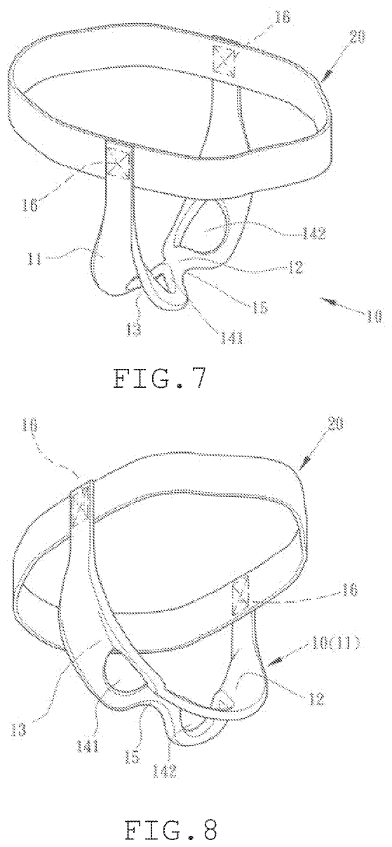

Referring to FIG. 1 to FIG. 8, the present invention provides a physiological fixed device described as a fixed device for females to use, which primarily includes a main body unit 10, an underclothing unit 20 connected with the main body unit 10, and at least one collecting bag 30 fixed on the main body unit 10.

The main body unit 10 has a main body 11, a predetermined part of which is V-shaped, and the main body 11 is respectively provided on top and bottom surfaces thereof with an abutted portion 12 for contacting a human body and an accommodating portion 13 forming a space. The abutted portion 12 is provided with an excretion collecting opening 14 with penetrating shape for excretion to fall into a collecting part. There are two excretion collecting openings 14 provided at two parts 141 and 142 of the main body unit 10 respectively in a way that the excretion collecting openings 141 and 142 correspond to the anus and the urethral orifice of the human body, respectively. The main body unit 10 is formed on each of the left and right sides thereof with a concave portion 15 having a preset concave arc surface for corresponding to the thighs located at the crotch of the human body. The main body unit 10 has two predetermined parts each integrally extending for a predetermined length and provided thereon with a sewing portion 16.

The underclothing unit 20 is connected with the main body unit 10. The underclothing unit 20 can be positioned on the human body and make the main body unit located at the crotch of the human body. The underclothing unit 20 has predetermined parts sewed on the sewing portions 16. The underclothing unit 20 may be an application of one of the types of a band, a belt, pants, culottes, a thong and a panty. This embodiment takes the type of a thong for description.

The collecting bags 30 are disposed at the excretion collecting openings 141 and 142 respectively for collecting the excretion therein.

By the aforementioned members, the abutted portion 12 with V-shaped cross sections can correspond to the physiological structure of the buttocks groove to be effectively fit, thereby having relatively better fitness, and the accommodating portion 13 is formed with a space for accommodating the collecting bag 30. In coordination with the underclothing unit 20, it is convenient for wearing and carrying, suitable for males and females with different physiological structures, useful for collecting feces and urine at the same time, and high in compatibility, so the entire structure is convenient and practical.

For further understanding of the structural features, used technical manners and expected effects of the present invention, the use method of the present invention is specified in the following.

For the assembly of the present invention, at first, the main body unit 10 is an elongated seat, which may be an application of one of the materials of silicone rubber, plastic material, paper and cloth. The main body unit 10 is integrally formed at the middle section thereof with the abutted portion 12, accommodating portion 13, two excretion collecting openings 141 and 142 and concave portions 15, which have V-shaped cross sections. The main body unit 10 is integrally formed at two ends of the elongated seat with the sewing portions 16, respectively. The underclothing unit 20 is shaped as an annular belt, which may be an application of a flexible band or elastic band. Fixedly and respectively connecting the predetermined parts of the belt-type underclothing unit 20 with the sewing portions 16 completes the physiological fixed device worn in the form of a thong.

For the structure of each collecting bag, referring to FIG. 17, which includes a bag body 301 having a space 33. The bag body 301 is provided at the bag opening thereof with at least one second positioning portion 32 to provide an engageable part. The second positioning portion 32 is formed in a way that a rope 34 is circularly disposed and positioned at the bag opening of the bag body 301 and hidden therein, and the partial rope 34 located at a predetermined part is exposed to form the engageable part. The rope 34 can be pulled to bind up the bag opening of the bag body 301. Alternatively, the exposed part of the rope 34 can be pulled to bind up the bag opening of the bag body 301.

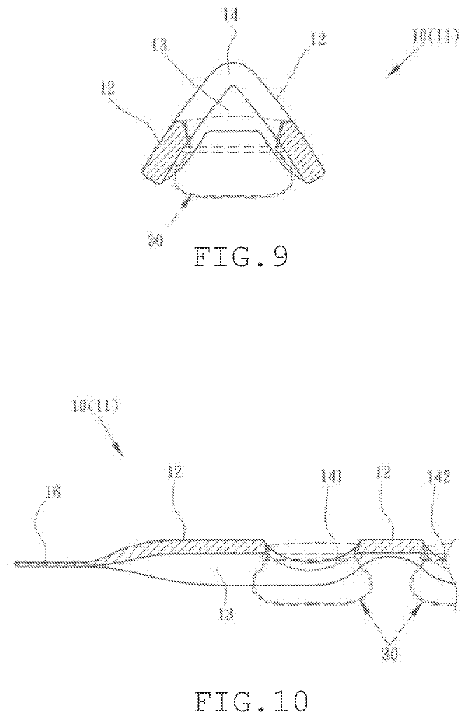

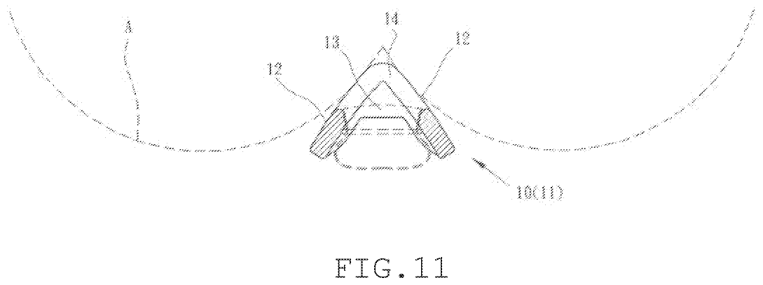

For the use of the present invention by wearing, referring to FIG. 9 to FIG. 10, at first, the collecting bags 30 are respectively fastened at the excretion collecting openings 141 and 142 in any form, the fastening manner of which includes embedding, hooking, adhering, or any detachable fastening manner, and the collecting bags 30 are located in the space of the accommodating portion 13. The physiological fixed device of the present invention assembled with the collecting bags 30 is worn on the crotch. Referring to FIG. 11, at this time the buttocks groove of the buttocks (A) of the human body is abutted on two sides of the abutted portion 12, the thighs of the human body are located at the concave portions 15, the excretion collecting openings 141 and 142 are located correspondingly to the urethral orifice and the anus of the human body respectively, and the wearing is accomplished.

For the present invention, when excretion exists, no matter it is feces, urine or menstrual blood, it directly flows into or enters the associated collecting bag 30. Because the main body 11 with V-shaped cross sections form the effectively supportive accommodating portion 13, it has quite space to prevent the collecting bags 30 already accommodating the excretion from being compressed.

The replacement of the present invention is performed by taking off the physiological fixed device, removing the collecting bag 30 accommodating the excretion, cleaning the main body unit 10 appropriately, and then installing an unused collecting bag 30 on the main body unit 10 in the manner as described above. The replacement will be accomplished by letting the patient wear the physiological fixed device again after the urethra or anus thereof is cleaned.

The present invention is provided with the elastic portion 21 to be adapted for a specific range of waist size, thereby having the function of elastic adjustment and effectively improving the wearing comfortableness. Meanwhile, according to different sex or requirement, the excretion collecting openings 141 and 142 can be modified in shape, size, distance and corresponding position, thereby having the function of elastic adjustment.

In the present invention, the main body 11 with V-shaped cross sections is used, which is provided thereon with the abutted portion 12, accommodating portion 13, excretion collecting openings 14, concave portions 15 and sewing portions 16, and the sewing portions 16 are used to be connected with the underclothing unit 20 to form the physiological fixed device, that has the following effects.

First, the main body 11 of the present invention has V-shaped cross sections, and the outer edge surface is an abutted portion 12 for fitting the buttocks groove. For conforming to the structure of the buttocks groove of the human body, it is relatively better in fitness, effective in improving comfortableness, and can solve the problem of the conventional structure that the poor fitness causes friction or strange walking posture.

Second, the main body 11 with V-shaped cross sections forms the space of the accommodating portion 13 to provide an accommodating space for the installation of the collecting bags 30 and thereby has the accommodating function. Besides, the accommodating portion 13 is quite supportive to prevent the collecting bag 30 already accommodating the excretion from being compressed, thereby reducing the situation that the excretion is compressed to leak out.

Third, the present invention combines the main body unit 10 with the underclothing unit 20, and makes the underclothing unit 20 provide the function of being worn and positioned on the human body, attaining the effect of convenience for wearing and carrying. The underclothing unit 20 may be an application of one of the types of a band, a belt, pants, culottes, a thong and a panty, having wide applicable range and high practicability.

Fourth, the present invention is suitable for males and females with different physiological structures, and useful for collecting feces, urine or menstrual blood at the same time, thereby having high compatibility in usage, high practicability and wide applicable range, solving the problems of the conventional technique that is mostly adapted for a single sex or collecting a single kind of excretion and does not have compatibility.

Fifth, the installation and replacement of the collecting bag 30 is simple and easy in the present invention. The collecting bag 30 is a consumable item and discarded when used up. The main body unit 10 and underclothing unit 20 are reusable and easy to clean, effectively attaining the environmental protection effect of waste reduction.

Sixth, the structure of the concave portion 15 is designed according to the thighs of the physiological structure of the crotch of the human body to form a space for extension to effectively reduce the friction with the thighs and improve the wearing comfortableness.

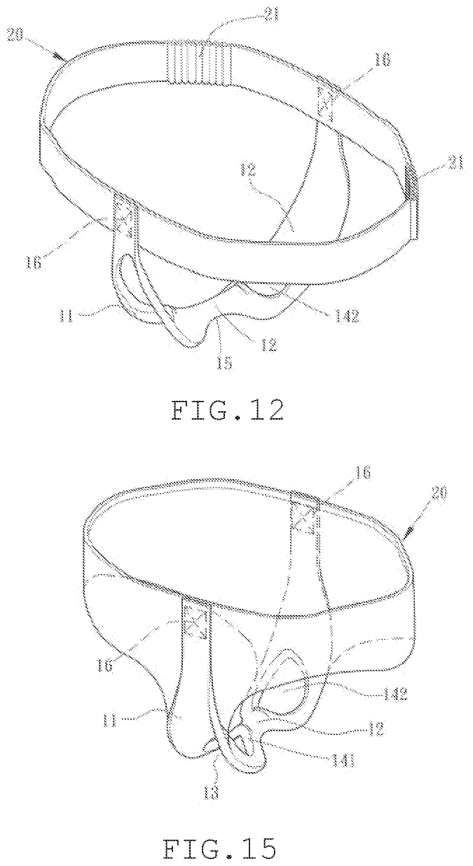

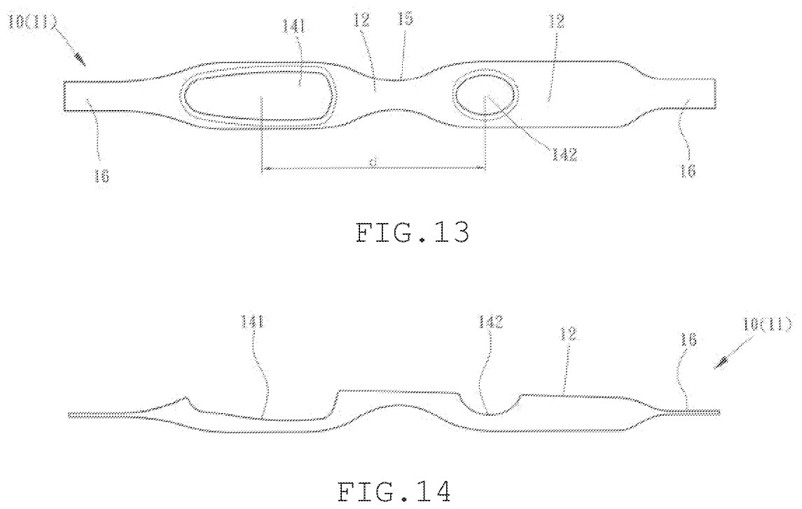

Referring to FIG. 12 to FIG. 14, a second embodiment of the present invention is the type for male physiological structure, wherein the underclothing unit 20 is provided at a predetermined part thereof with an elastic portion 21 for elastic adjustment of waist sizes of different body shapes at wearing parts. According to the male physiological structure, the distance d between the excretion collecting openings 141 and 142 is sized relatively longer. The excretion collecting opening 14 corresponding to the male genitalia is required with relatively larger hollow notch, and its position is also offset toward the abdomen to conform to the physiological structure of the human body better.

Referring to FIG. 15, a third embodiment of the present invention is the type providing the underclothing unit 20 in the form of a panty. The use state, spatial form, component composition, acting manner and expected effect of this embodiment are exactly the same with those of the above-described first embodiment.

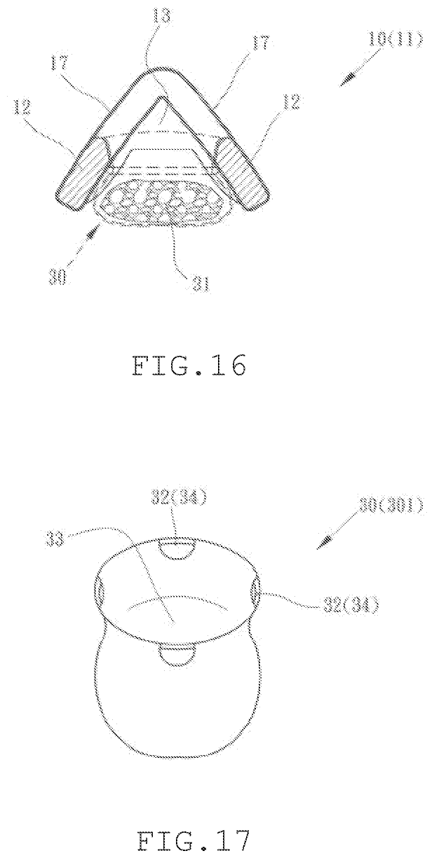

Referring to FIG. 16, in a fourth embodiment of the present invention, the main body unit 10 is covered with a cloth layer 17, making the main body unit 10 relatively more comfortable for abutting the skin of the human body. The collecting bag 30 is provided therein with an absorbing member 31 for absorbing liquid excretion. The absorbing member 31 may be an application of cotton, sponge or super absorbent polymer (SAP). The use state, spatial form, component composition, acting manner and expected effect of this embodiment are exactly the same with those of the above-described first embodiment.

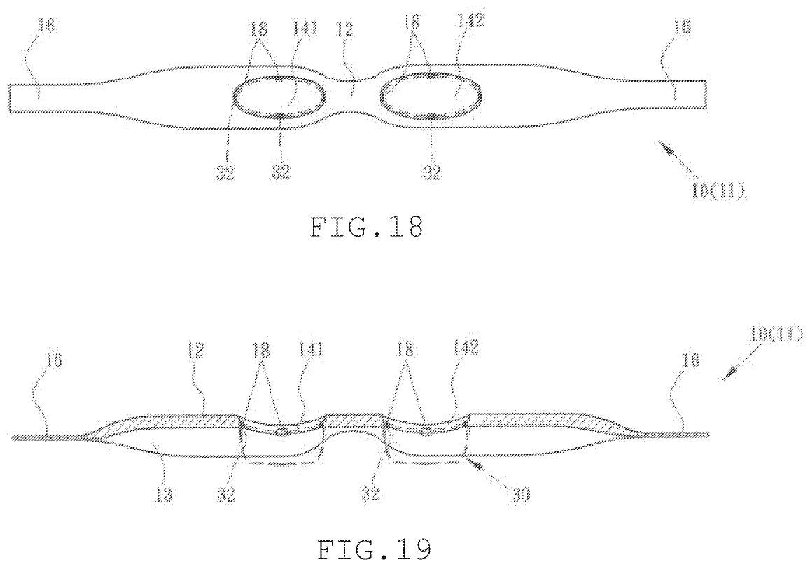

Referring to FIG. 17 to FIG. 19, in a fifth embodiment of the present invention, the excretion collecting opening 14 is provided with at least one first positioning portion 18, and the collecting bag 30 has at least one second positioning portion 32 fixedly connected with the first positioning portion 18. The part of the second positioning portion 32 connected with the first positioning portion 18 is a rope 34 which is also a binding member for the bag opening of the collecting bag 30. The second positioning portion 32 is formed in a way that a can body 34 is circularly disposed and positioned at the bag opening of the bag body 301 and hidden therein. The partial rope 34 located at a predetermined part is exposed to form the second positioning portion 32 for being hooked and engaged with the first positioning portion 18. When the collecting bag 30 is removed, pulling the exposed part of the rope 34 can bind up the bag opening of the bag body 301.

This embodiment provides another structure of the collecting bag 30 and the positioning structure thereof connected with the excretion collecting openings 141 and 142, that uses a hook manner to make the first and second positioning portions 18 and 32 hooked with each other to complete the fixation. For the replacement, after the collecting bag 30 is removed, pulling out the rope 34 located at the opening of the collecting bag 30 to gradually lengthen the exposed part can bind up the opening of the collecting bag 30 to close it. The use state, spatial form, component composition, acting manner and expected effect of this embodiment are exactly the same with those of the above-described first embodiment.

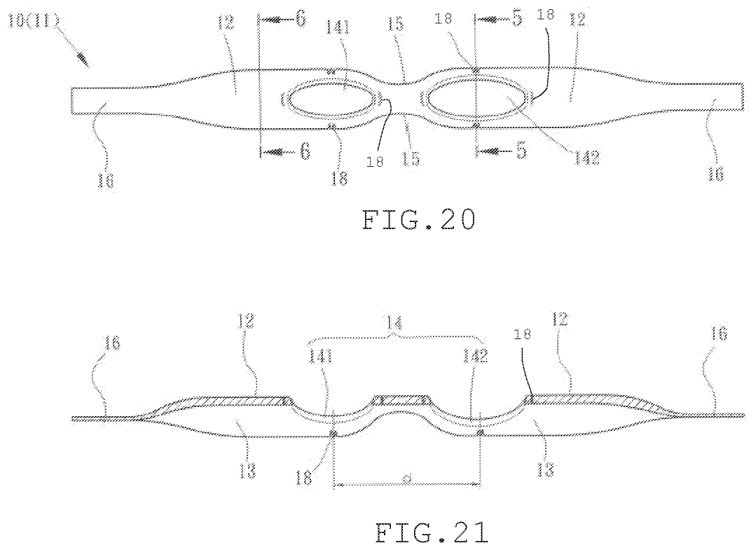

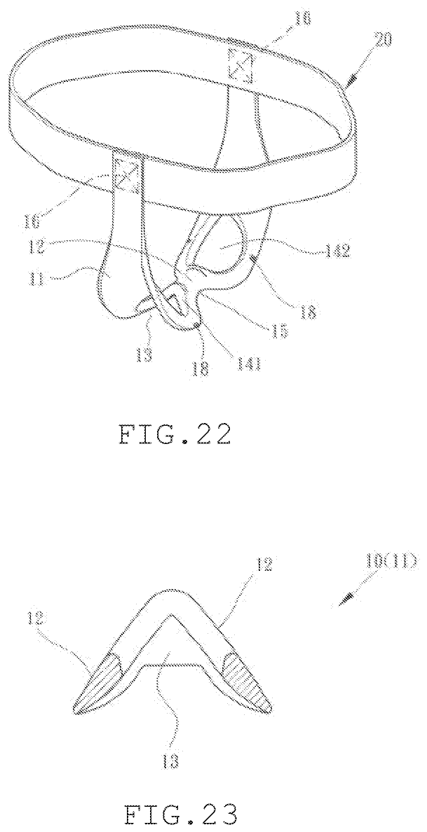

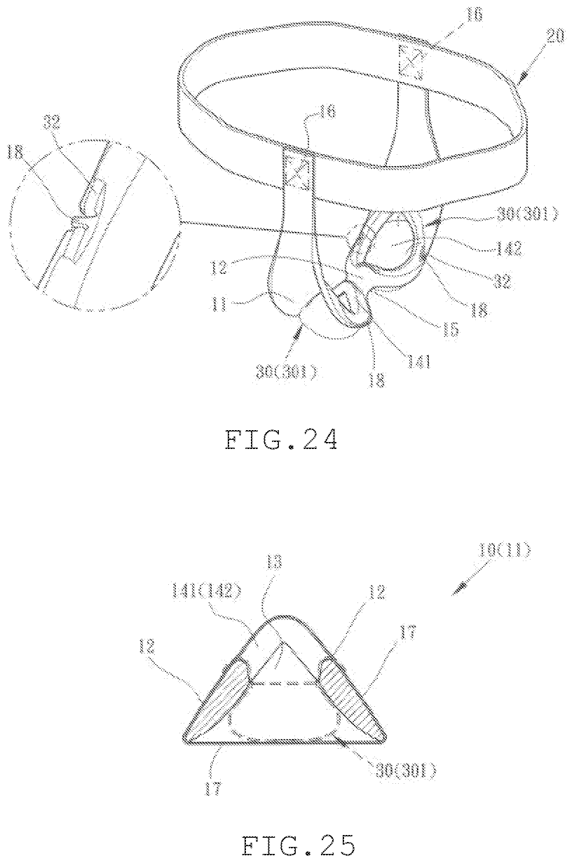

Referring to FIG. 20 to FIG. 24, a sixth embodiment of the present invention provides another type of engaging structure of the main body unit 10 and the collecting bag 30, wherein the main body unit 10 is formed at a predetermined part thereof with at least one first positioning portion 18, which may be a W-shaped double recess provided on a predetermined edge position of the main body unit 10. Its positioning engaging manner is to cooperate with the collecting bag 30 in the embodiment before in a way that the rope 34 of the second positioning portion 32 is hooked between the doble recess of the first positioning portion 18.

This embodiment provides another structure of the collecting bag 30 and the positioning structure thereof connected with predetermined parts located close to the excretion collecting openings 141 and 142, that uses a hook manner to make the first and second positioning portions 18 and 32 hooked with each other to complete the fixation. For the replacement, after the collecting bag 30 is removed, pulling out the rope located at the opening of the collecting bag 30 to gradually lengthen the exposed part can bind up the opening of the collecting bag 30 to close it. The use state, spatial form, component composition, acting manner and expected effect of this embodiment are exactly the same with those of the above-described first embodiment.

Wherein, the first positioning portion 18 may be a recess, a cut groove or a protruding column, and the second positioning portion 32 may be a ring buckle, an inverted T-shaped buckle or a hook-shaped buckle.

Referring to FIG. 25, in a seventh embodiment of the present invention, the main body unit 10 is covered with a cloth layer 17 in a way that the cloth layer 17 completely covers the accommodating portion 13 to make the collecting bag 30 hidden in the accommodating portion 13 and the cloth layer 17.

The cloth layer 17 in this embodiment is effective in hiding the collecting bag in the accommodating portion 13 and the cloth layer 17, thereby attaining the covering and aesthetic effects. The use state, spatial form, component composition, acting manner and expected effect of this embodiment are exactly the same with those of the above-described first embodiment.

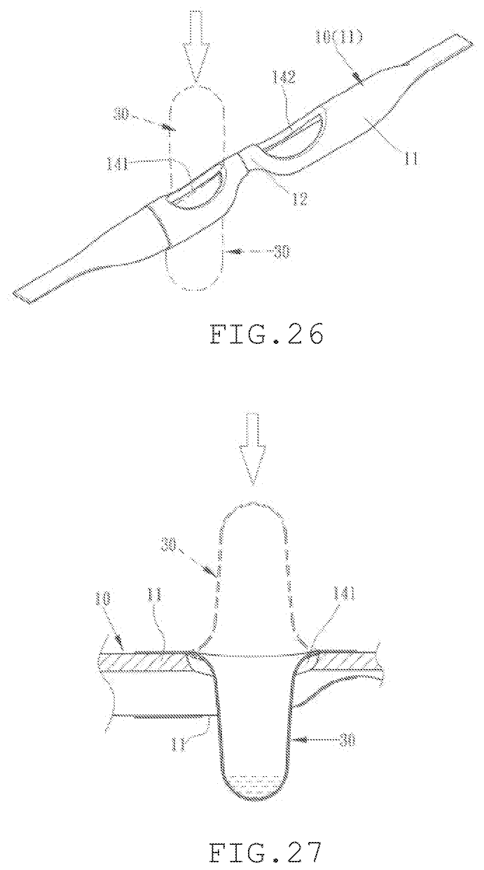

Referring to FIG. 26 to FIG. 27, an eighth embodiment of the present invention primarily provides another type of structure of the collecting bag 30 and the spatial form application of its connection with the main body unit 10, wherein the collecting bag 30 is disposed on the main body unit 10 in a sleeved manner, and after the collecting bag 30 is positioned correspondingly to the excretion collecting opening 141 or 142, the collecting bag 30 is retracted toward the excretion collecting opening 141 or 142 to form a first space for collecting the excretion. The use state, spatial form, component composition, acting manner and expected effect of this embodiment are exactly the same with those of the above-described first embodiment.

It can be known from the above disclosure of the embodiments that the collecting bag 30 of the present invention can be any type. The connecting positioning structure of the first and second positioning portions 18 and 32 can be attained by using any common structural design such as engaging or hooking.

In conclusion, the physiological fixed device disclosed in the present invention provides a physiological fixed device convenient for wearing and carrying, which combines the main body unit placed in the buttocks groove with the underclothing unit worn on the human body. The main body unit with V-shaped cross sections is provided according to the human body structure with the excretion collecting openings, concave portions and abutted portion, and also formed with the accommodating portion for accommodating the collecting bags. After the installation of the collecting bags at the excretion collecting openings, it is convenient to be worn and positioned or taken off for replacement, and it is relatively better in fitness, suitable for males and females with different physiological structures, and useful for collecting feces and urine at the same time, so that it is a physiological fixed and nursing structure with high compatibility and practicality. That makes the entirety indeed have industrial practicality and cost benefit, and its structure has not been seen in books or public use, thereby in compliance with the requirements for patent application. Sincerely and gratefully ask for the approvement of the patent soon.

It should be mentioned that the above description is the concrete embodiments of the present invention and the technical theorem used. Any modification made according to the concept of the present invention and producing the functions and effects not departed from the spirit included in the specification and the figures should be regarded within the scope of the present invention.

What is claimed is:

1. A physiological fixed device comprising:
a main body unit comprising a main body provided at a predetermined part thereof with V-shaped cross sections, the main body being respectively provided on top and bottom surfaces thereof with an abutted portion for contacting a human body and an accommodating portion forming a space, the abutted portion being provided with at least one excretion collecting opening with penetrating shape for excretion to fall into a collecting part.

2. The physiological fixed device as claimed in claim 1, wherein the main body unit is formed on each of left and right sides thereof with a concave portion having a preset concave arc surface.

3. The physiological fixed device as claimed in claim 1, wherein the physiological fixed device comprises an underclothing unit connected with the main body unit; the underclothing unit is adapted to be positioned on the human body and make the main body unit located at a crotch of the human body.

4. The physiological fixed device as claimed in claim 1, wherein the main body unit is provided at a predetermined part thereof with at least one sewing portion; an underclothing unit is sewed on the sewing portion; the underclothing unit is adapted to be positioned on the human body and make the main body unit located at a crotch of the human body.

5. The physiological fixed device as claimed in claim 4, wherein the underclothing unit is provided at a predetermined part thereof with an elastic portion for elastic adjustment of different body sizes at wearing parts.

6. The physiological fixed device as claimed in claim 1, wherein the main body unit is covered with a cloth layer.

7. The physiological fixed device as claimed in claim 1, wherein two said excretion collecting openings are provided at two parts of the main body unit respectively in a way that the excretion collecting openings correspond to an anus and a urethral orifice of the human body respectively.

8. The physiological fixed device as claimed in claim 1, wherein the main body unit is an application of one of materials of silicone rubber, plastic material, paper and cloth.

9. An assembly of the physiological fixed device as claimed in claim 1 and a collecting bag, wherein the collecting bag is disposed at the excretion collecting opening for collecting the excretion therein.

10. The assembly of the physiological fixed device and the collecting bag as claimed in claim 9, wherein the collecting bag is provided therein with an absorbing member for absorbing liquid excretion.

11. The assembly of the physiological fixed device and the collecting bag as claimed in claim 10, wherein the absorbing member is an application of one of cotton, sponge and super absorbent polymer (SAP).

12. The assembly of the physiological fixed device and the collecting bag as claimed in claim 9, wherein the main body unit is covered with a cloth layer in a way that the cloth layer completely covers the accommodating portion to make the collecting bag hidden in the accommodating portion and the cloth layer.

13. The assembly of the physiological fixed device and the collecting bag as claimed in claim 9, wherein the main body unit is formed at a predetermined part thereof with at least one first positioning portion; the collecting bag comprises a bag body and at least one second positioning portion connected and positioned with the first positioning portion.

14. The assembly of the physiological fixed device and the collecting bag as claimed in claim 13, wherein the second positioning portion is formed in a way that a rope is circularly disposed and positioned at a bag opening of the bag body and hidden therein and the partial rope located at a predetermined part is exposed to form the second positioning portion.

15. The assembly of the physiological fixed device and the collecting bag as claimed in claim 13, wherein the bag body has a rope for being pulled to bind up a bag opening of the bag body.

16. The assembly of the physiological fixed device and the collecting bag as claimed in claim 13, wherein the first positioning portion is one of a recess, a cut groove and a protruding column.

17. The assembly of the physiological fixed device and the collecting bag as claimed in claim 13, wherein the second positioning portion is one of a ring buckle, an inverted T-shaped buckle and a hook-shaped buckle.

18. The assembly of the physiological fixed device and the collecting bag as claimed in claim 9, wherein the collecting bag comprises a bag body having a space; the bag body is provided at a bag opening thereof with at least one second positioning portion for providing an engageable part.
